(19) United States
(12) Patent Application Publication
(10) Pub. No.: US 2008/0319086 A1
(43) Pub. Date: Dec. 25, 2008

(54) DEUTERIUM-ENRICHED SALMETEROL

(75) Inventor: Anthony W. Czarnik, Reno, NV (US)

Correspondence Address: VANCE INTELLECTUAL PROPERTY, PC, 5467 HILL TOP STREET, CROZET, VA, 22932-3167, US

(73) Assignee: PROTIA, LLC, Reno, NV (US)

(21) Appl. No.: 11/766,080

(22) Filed: Jun. 20, 2007

(51) Int. Cl. A61K 31/135; A61P 11/06; C07C 217/02

(52) U.S. Cl. 514/651; 564/355

(57) ABSTRACT

The present application describes deuterium-enriched salmeterol, pharmaceutically acceptable salt forms thereof, and methods of treating using the same.

### FIELD OF THE INVENTION

This invention relates generally to deuterium-enriched salmeterol, pharmaceutical compositions containing the same, and methods of using the same.

### BACKGROUND OF THE INVENTION

Salmeterol, shown below, is a well known beta2-adrenergic receptor agonist.

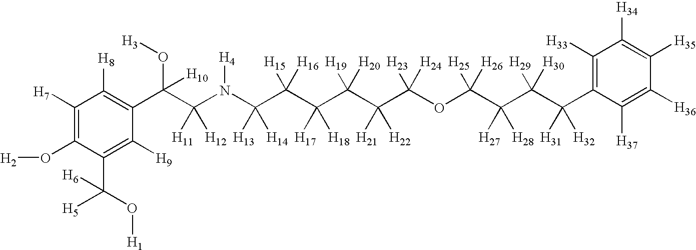

Since salmeterol is a known and useful pharmaceutical, it is desirable to discover novel derivatives thereof. Salmeterol is described in U.S. Pat. No. 4,992,474; the contents of which are incorporated herein by reference.

### SUMMARY OF THE INVENTION

Accordingly, one object of the present invention is to provide deuterium-enriched salmeterol or a pharmaceutically acceptable salt thereof.

It is another object of the present invention to provide pharmaceutical compositions comprising a pharmaceutically acceptable carrier and a therapeutically effective amount of at least one of the deuterium-enriched compounds of the present invention or a pharmaceutically acceptable salt thereof.

It is another object of the present invention to provide a method for treating asthma, comprising administering to a host in need of such treatment a therapeutically effective amount of at least one of the deuterium-enriched compounds of the present invention or a pharmaceutically acceptable salt thereof.

It is another object of the present invention to provide a novel deuterium-enriched salmeterol or a pharmaceutically acceptable salt thereof for use in therapy.

It is another object of the present invention to provide the use of a novel deuterium-enriched salmeterol or a pharmaceutically acceptable salt thereof for the manufacture of a medicament (e.g., for the treatment of asthma).

These and other objects, which will become apparent during the following detailed description, have been achieved by the inventor's discovery of the presently claimed deuterium-enriched salmeterol.

### DETAILED DESCRIPTION OF PREFERRED EMBODIMENTS

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their non-enriched counterparts.

All percentages given for the amount of deuterium present are mole percentages.

It can be quite difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

The present invention provides deuterium-enriched salmeterol or a pharmaceutically acceptable salt thereof There are thirty-seven hydrogen atoms in the salmeterol portion of salmeterol as show by variables R₁-R₃₇ in formula I below.

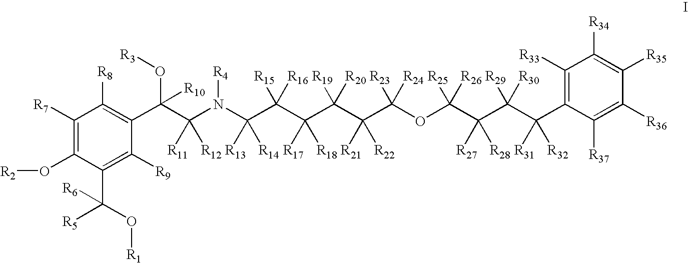

The hydrogens present on salmeterol have different capacities for exchange with deuterium. Hydrogen atoms R₁-R₄ are easily exchangeable under physiological conditions and, if replaced by deuterium atoms, it is expected that they will readily exchange for protons after administration to a patient. The remaining hydrogen atoms are not easily exchangeable and may be incorporated by the use of deuterated starting materials or intermediates during the construction of salmeterol. Salmeterol with R₅-R₆ and R₁₀=D is known, though not for therapeutic applications. See Molinski, et al., J. Label. Cpd. Radiopharm. 2002, 45, 755-762.

The present invention is based on increasing the amount of deuterium present in salmeterol above its natural abundance. This increasing is called enrichment or deuterium-enrichment. If not specifically noted, the percentage of enrichment refers to the percentage of deuterium present in the compound, mixture of compounds, or composition. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Since there are 37 hydrogens in salmeterol, replacement of a single hydrogen atom with deuterium would result in a molecule with about 3% deuterium enrichment. In order to achieve enrichment less than about 3%, but above the natural abundance, only partial deuteration of one site is required. Thus, less than about 3% enrichment would still refer to deuterium-enriched salmeterol.

With the natural abundance of deuterium being 0.015%, one would expect that for approximately every 6,667 molecules of salmeterol (1/0.00015=6,667), there is one naturally occurring molecule with one deuterium present. Since salmeterol has 37 positions, one would roughly expect that for approximately every 246,679 molecules of salmeterol (37×6,667), all 37 different, naturally occurring, mono-deuterated salmeterols would be present. This approximation is a rough estimate as it doesn't take into account the different exchange rates of the hydrogen atoms on salmeterol. For naturally occurring molecules with more than one deuterium, the numbers become vastly larger. In view of this natural abundance, the present invention, in an embodiment, relates to an amount of an deuterium enriched compound, whereby the enrichment recited will be more than naturally occurring deuterated molecules.

In view of the natural abundance of deuterium-enriched salmeterol, the present invention also relates to isolated or purified deuterium-enriched salmeterol. The isolated or purified deuterium-enriched salmeterol is a group of molecules whose deuterium levels are above the naturally occurring levels (e.g., 3%). The isolated or purified deuterium-enriched salmeterol can be obtained by techniques known to those of skill in the art (e.g., see the syntheses described below).

The present invention also relates to compositions comprising deuterium-enriched salmeterol. The compositions require the presence of deuterium-enriched salmeterol which is greater than its natural abundance. For example, the compositions of the present invention can comprise (a) a μg of a deuterium-enriched salmeterol; (b) a mg of a deuterium-enriched salmeterol; and, (c) a gram of a deuterium-enriched salmeterol.

In an embodiment, the present invention provides an amount of a novel deuterium-enriched salmeterol.

Examples of amounts include, but are not limited to (a) at least 0.01, 0.02, 0.03, 0.04, 0.05, 0.1, 0.2, 0.3, 0.4, 0.5, to 1 mole, (b) at least 0.1 moles, and (c) at least 1 mole of the compound. The present amounts also cover lab-scale (e.g., gram scale), kilo-lab scale (e.g., kilogram scale), and industrial or commercial scale (e.g., multi-kilogram or above scale) quantities as these will be more useful in the actual manufacture of a pharmaceutical. Industrial/commercial scale refers to the amount of product that would be produced in a batch that was designed for clinical testing, formulation, sale/distribution to the public, etc.

In another embodiment, the present invention provides a novel, deuterium enriched compound of formula I or a pharmaceutically acceptable salt thereof.

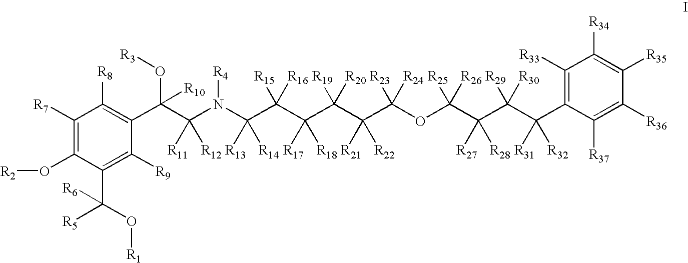

wherein R₁-R₃₇ are independently selected from H and D; and the abundance of deuterium in R₁-R₃₇ is at least 3%, provided that if R₅-R₆ and R₁₀ are D, then at least one other R is a D. The abundance can also be (a) at least 5%, (b) at least 11%, (c) at least 16%, (d) at least 22%, (e) at least 27%, (f) at least 32%, (g) at least 38%, (h) at least 43%, (i) at least 49%, (j) at least 54%, (k) at least 59%, (l) at least 65%, (m) at least 70%, (n) at least 76%, (o) at least 81%, (p) at least 86%, (q) at least 92%, (r) at least 97%, and (s) 100%.

In another embodiment, the present invention provides a novel, deuterium enriched compound of formula I or a pharmaceutically acceptable salt thereof, wherein the abundance of deuterium in R₁-R₄ is at least 25%. The abundance can also be (a) at least 50%, (b) at least 75%, and (c) 100%.

In another embodiment, the present invention provides a novel, deuterium enriched compound of formula I or a pharmaceutically acceptable salt thereof, wherein the abundance of deuterium in R₅-R₆ is at least 50%. The abundance can also be (a) 100%.

In another embodiment, the present invention provides a novel, deuterium enriched compound of formula I, wherein the abundance of deuterium in R₇-R₉ is at least 33%. The abundance can also be (a) at least 67%, and (b) 100%.

In another embodiment, the present invention provides a novel, deuterium enriched compound of formula I or a pharmaceutically acceptable salt thereof, wherein the abundance of deuterium in R₁₀-R₁₂ is at least 33%. The abundance can also be (a) at least 67%, and (b) 100%.

In another embodiment, the present invention provides a novel, deuterium enriched compound of formula I or a pharmaceutically acceptable salt thereof, wherein the abundance of deuterium in R₁₃-R₂₄ is at least 8%. The abundance can also be (a) at least 17%, (b) at least 25%, (c) at least 33%, (d) at least 42%, (e) at least 50%, (f) at least 58%, (g) at least 67%, (h) at least 75%, (i) at least 83%, (j) at least 92%, and (k) 100%.

In another embodiment, the present invention provides a novel, deuterium enriched compound of formula I or a pharmaceutically acceptable salt thereof, wherein the abundance of deuterium in R₂₅-R₃₂ is at least 13%. The abundance can also be (a) at least 25%, (b) at least 38%, (c) at least 50%,(d) at least 63%, (e) at least 75%, (f) at least 88%, and (g) 100%.

In another embodiment, the present invention provides a novel, deuterium enriched compound of formula I or a pharmaceutically acceptable salt thereof, wherein the abundance of deuterium in R₃₃-R₃₇ is at least 20%. The abundance can also be (a) at least 40%, (b) at least 60%, (c) at least 80%, and (d) 100%.

In another embodiment, the present invention provides a novel, isolated deuterium enriched compound of formula I or a pharmaceutically acceptable salt thereof.

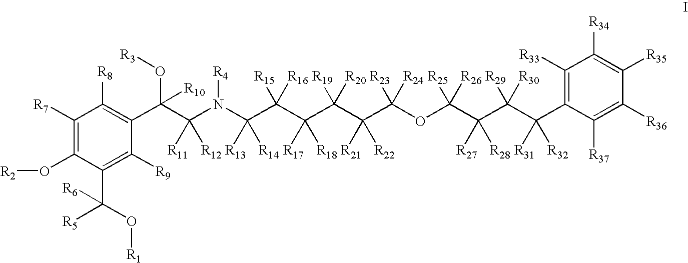

wherein R₁-R₃₇ are independently selected from H and D; and the abundance of deuterium in R₁-R₃₇ is at least 3%, provided that if R₅-R₆ and R₁₀ are D, then at least one other R is a D. The abundance can also be (a) at least 5%, (b) at least 11%, (c) at least 16%, (d) at least 22%, (e) at least 27%, (f) at least 32%, (g) at least 38%, (h) at least 43%, (i) at least 49%, (j) at least 54%, (k) at least 59%, (l) at least 65%, (m) at least 70%, (n) at least 76%, (o) at least 81%, (p) at least 86%, (q) at least 92%, (r) at least 97%, and (s) 100%.

In another embodiment, the present invention provides a novel, isolated deuterium enriched compound of formula I or a pharmaceutically acceptable salt thereof, wherein the abundance of deuterium in R₁-R₄ is at least 25%. The abundance can also be (a) at least 50%, (b) at least 75%, and (c) 100%.

In another embodiment, the present invention provides a novel, isolated deuterium enriched compound of formula I or a pharmaceutically acceptable salt thereof, wherein the abundance of deuterium in R₅-R₆ is at least 50%. The abundance can also be (a) 100%.

In another embodiment, the present invention provides a novel, isolated deuterium enriched compound of formula I, wherein the abundance of deuterium in R₇-R₉ is at least 33%. The abundance can also be (a) at least 67%, and (b) 100%.

In another embodiment, the present invention provides a novel, isolated deuterium enriched compound of formula I or a pharmaceutically acceptable salt thereof, wherein the abundance of deuterium in R₁₀-R₁₂ is at least 33%. The abundance can also be (a) at least 67%, and (b) 100%.

In another embodiment, the present invention provides a novel, isolated deuterium enriched compound of formula I or a pharmaceutically acceptable salt thereof, wherein the abundance of deuterium in R₁₃-R₂₄ is at least 8%. The abundance can also be (a) at least 17%, (b) at least 25%, (c) at least 33%, (d) at least 42%, (e) at least 50%, (f) at least 58%, (g) at least 67%, (h) at least 75%, (i) at least 83%, (j) at least 92%, and (k) 100%.

In another embodiment, the present invention provides a novel, isolated deuterium enriched compound of formula I or a pharmaceutically acceptable salt thereof, wherein the abundance of deuterium in R₂₅-R₃₂ is at least 13%. The abundance can also be (a) at least 25%, (b) at least 38%, (c) at least 50%,(d) at least 63%, (e) at least 75%, (f) at least 88%, and (g) 100%.

In another embodiment, the present invention provides a novel, isolated deuterium enriched compound of formula I or a pharmaceutically acceptable salt thereof, wherein the abundance of deuterium in R₃₃-R₃₇ is at least 20%. The abundance can also be (a) at least 40%, (b) at least 60%, (c) at least 80%, and (d) 100%.

In another embodiment, the present invention provides novel mixture of deuterium enriched compounds of formula I or a pharmaceutically acceptable salt thereof.

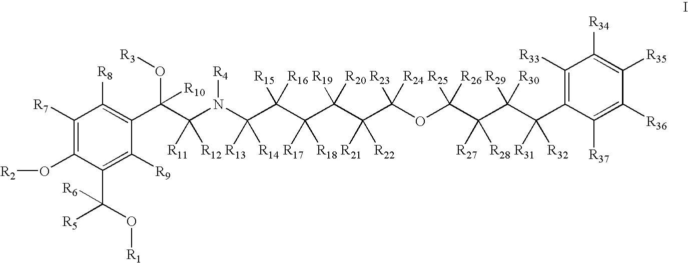

wherein R₁-R₃₇ are independently selected from H and D; and the abundance of deuterium in R₁-R₃₇ is at least 3%, provided that if R₅-R₆ and R₁₀ are D, then at least one other R is a D. The abundance can also be (a) at least 5%, (b) at least 11%, (c) at least 16%, (d) at least 22%, (e) at least 27%, (f) at least 32%, (g) at least 38%, (h) at least 43%, (i) at least 49%, (j) at least 54%, (k) at least 59%, (l) at least 65%, (m) at least 70%, (n) at least 76%, (o) at least 81%, (p) at least 86%, (q) at least 92%, (r) at least 97%, and (s) 100%.

In another embodiment, the present invention provides a novel mixture of deuterium enriched compound of formula I or a pharmaceutically acceptable salt thereof, wherein the abundance of deuterium in R₁-R₄ is at least 25%. The abundance can also be (a) at least 50%, (b) at least 75%, and (c) 100%.

In another embodiment, the present invention provides a novel mixture of deuterium enriched compound of formula I or a pharmaceutically acceptable salt thereof, wherein the abundance of deuterium in R₅-R₆ is at least 50%. The abundance can also be (a) 100%.

In another embodiment, the present invention provides a novel mixture of deuterium enriched compound of formula I, wherein the abundance of deuterium in R₇-R₉ is at least 33%. The abundance can also be (a) at least 67%, and (b) 100%.

In another embodiment, the present invention provides a novel mixture of deuterium enriched compound of formula I or a pharmaceutically acceptable salt thereof, wherein the abundance of deuterium in R₁₀-R₁₂ is at least 33%. The abundance can also be (a) at least 67%, and (b) 100%.

In another embodiment, the present invention provides a novel mixture of deuterium enriched compound of formula I or a pharmaceutically acceptable salt thereof, wherein the abundance of deuterium in R₁₃-R₂₄ is at least 8%. The abundance can also be (a) at least 17%, (b) at least 25%, (c) at least 33%, (d) at least 42%, (e) at least 50%, (f) at least 58%, (g) at least 67%, (h) at least 75%, (i) at least 83%, (j) at least 92%, and (k) 100%.

In another embodiment, the present invention provides a novel mixture of deuterium enriched compound of formula I or a pharmaceutically acceptable salt thereof, wherein the abundance of deuterium in R₂₅-R₃₂ is at least 13%. The abundance can also be (a) at least 25%, (b) at least 38%, (c) at least 50%,(d) at least 63%, (e) at least 75%, (f) at least 88%, and (g) 100%.

In another embodiment, the present invention provides a novel mixture of deuterium enriched compound of formula I or a pharmaceutically acceptable salt thereof, wherein the abundance of deuterium in R₃₃-R₃₇ is at least 20%. The abundance can also be (a) at least 40%, (b) at least 60%, (c) at least 80%, and (d) 100%.

In another embodiment, the present invention provides novel pharmaceutical compositions, comprising: a pharmaceutically acceptable carrier and a therapeutically effective amount of a deuterium-enriched compound of the present invention.

In another embodiment, the present invention provides a novel method for treating asthma comprising: administering to a patient in need thereof a therapeutically effective amount of a deuterium-enriched compound of the present invention.

In another embodiment, the present invention provides an amount of a deuterium-enriched compound of the present invention as described above for use in therapy.

In another embodiment, the present invention provides the use of an amount of a deuterium-enriched compound of the present invention for the manufacture of a medicament (e.g., for the treatment of asthma).

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

### DEFINITIONS

The examples provided in the definitions present in this application are non-inclusive unless otherwise stated. They include but are not limited to the recited examples.

The compounds of the present invention may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. All processes used to prepare compounds of the present invention and intermediates made therein are considered to be part of the present invention. All tautomers of shown or described compounds are also considered to be part of the present invention.

“Host” preferably refers to a human. It also includes other mammals including the equine, porcine, bovine, feline, and canine families.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antiviral effect, or some other beneficial effect of the combination compared with the individual components.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

### Synthesis

Scheme 1 shows a route to salmeterol (a combination of chemistry from Skidmore, et al., U.S. Pat. No. 4,992,474, Evans, P. EP 0422889 A2 1990, and Molinski, et al., J. Lab. Cpd. Radiopharm. 2002, 45, 755-762).

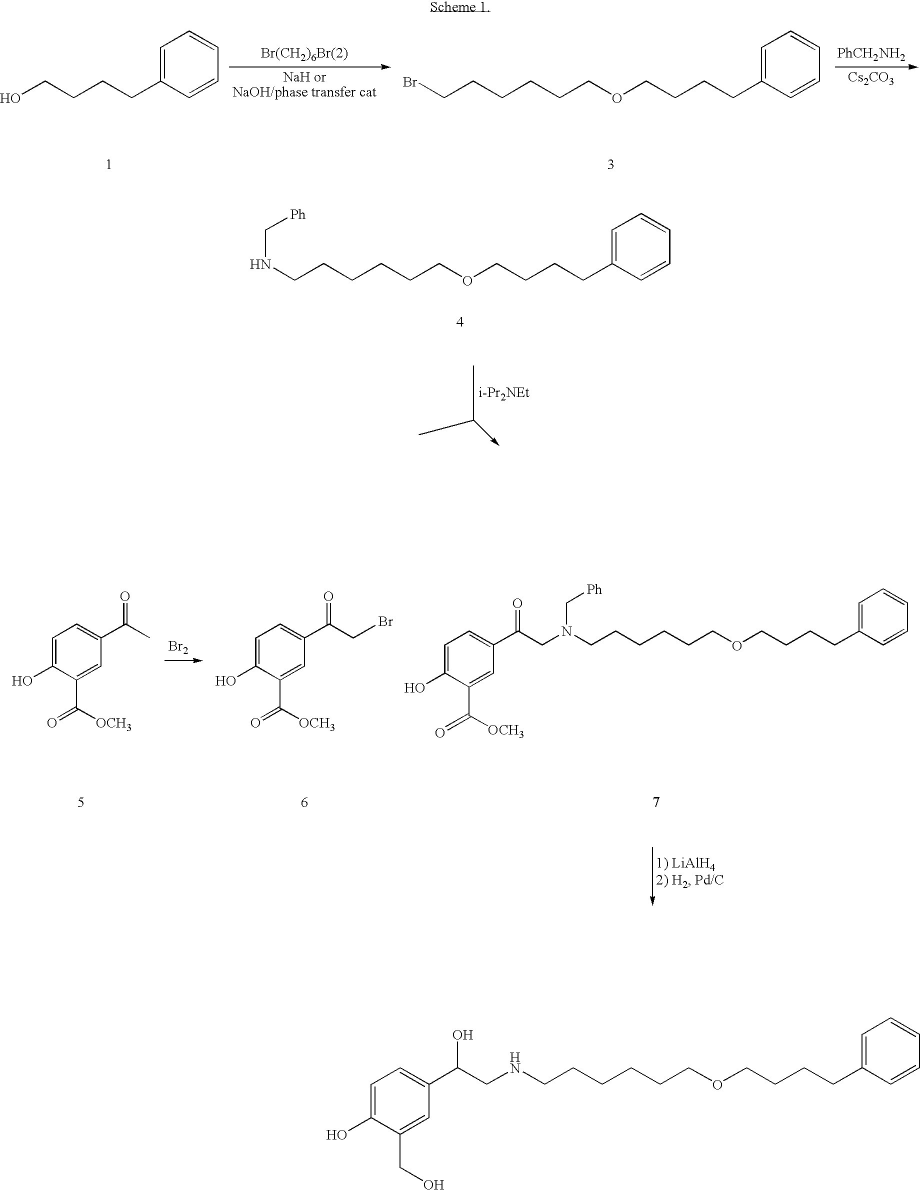

Schemes 2 and 3 show how various deuterated starting materials and intermediates can be used in the chemistry of Scheme 1 to make deuterated salmeterol analogs. A person skilled in the art of organic synthesis will recognize that these materials may be used in various combinations to access many other deuterated salmeterols that are not shown. Bromination of 1 followed by deuterolysis as shown in equation (1) of Scheme 2 provides 2 or 3, depending on how the bromination reaction is run. If 2 is used in the chemistry of Scheme 1 in place of 1, salmeterol with R₃₅=D results. If 3 is used in the chemistry of Scheme 1 in place of 1, salmeterol with R₃₃ and R₃₅=D results. Other deuterated forms of 1 are known, i.e., 4-7. If 4 is used in the chemistry of Scheme 1 in place of 1, salmeterol with R₂₇-R₃₂=D results. If 5 is used in the chemistry of Scheme 1 in place of 1, salmeterol with R₂₅-R₂₆=D results. If 6 is used in the chemistry of Scheme 1 in place of 1, salmeterol with R₃₁-R₃₂=D results. If 7 is used in the chemistry of Scheme 1 in place of 1, salmeterol with R₂₇-R₂₈=D results. Other deuterated forms of 1 may be prepared based on the chemistry of equation (2) of Scheme 2. For the all-proton version of this sequence, see Jiang, et al., Chem. Abstr. 2003, 141: 6877. Using hexadeuteriobenzene as shown in equations (3)-(6), compound 10 and thus compounds 11-14 may be made. If 11 is used in the chemistry of Scheme 1 in place of 1, salmeterol with R₃₃-R₃₇=D results. If 12 is used in the chemistry of Scheme 1 in place of 1, salmeterol with R₃₃-R₃₇, R₂₅-R₂₆, and R31-R32=D results. If 13 is used in chemistry of Scheme 1 in place of 1, salmeterol with R₃₃-R₃₇ and R₃₁-R₃₂=D results. If 14 is used in the chemistry of Scheme 1 in place of 1, salmeterol with R₃₃-R₃₇ and R₂₅-R₂₆=D results. Hydrogen-deuterium exchange on 8 affords 15, which may be used to make 16-18 as shown in equations (7)-(10). If 16 is used in the chemistry of Scheme 1 in place of 1, salmeterol with R₂₇-R₃₀=D results. If 17 is used in the chemistry of Scheme 1 in place of 1, salmeterol with R₂₅-R₃₂=D results. If 18 is used in the chemistry of Scheme 1 in place of 1, salmeterol with R₂₅-R₃₀=D results. Combinations of the above processes can be used to make many other compounds. For example, not wishing to be limited to a single example, 10 can be transformed into 19 according to equation (1). If 19 is used in the chemistry of Scheme 1 in place of 1, salmeterol with R₂₅-R₃₇=D results.

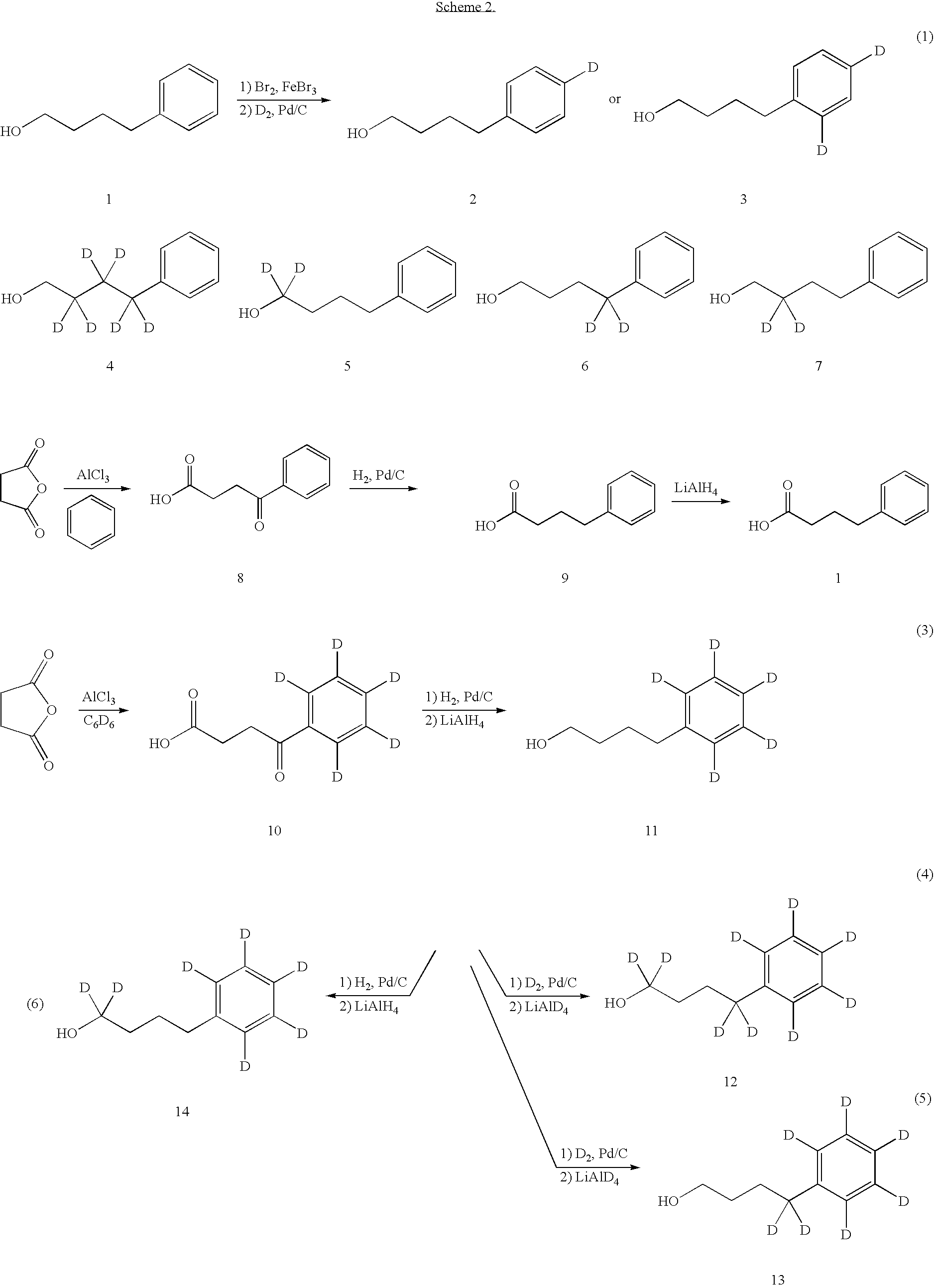

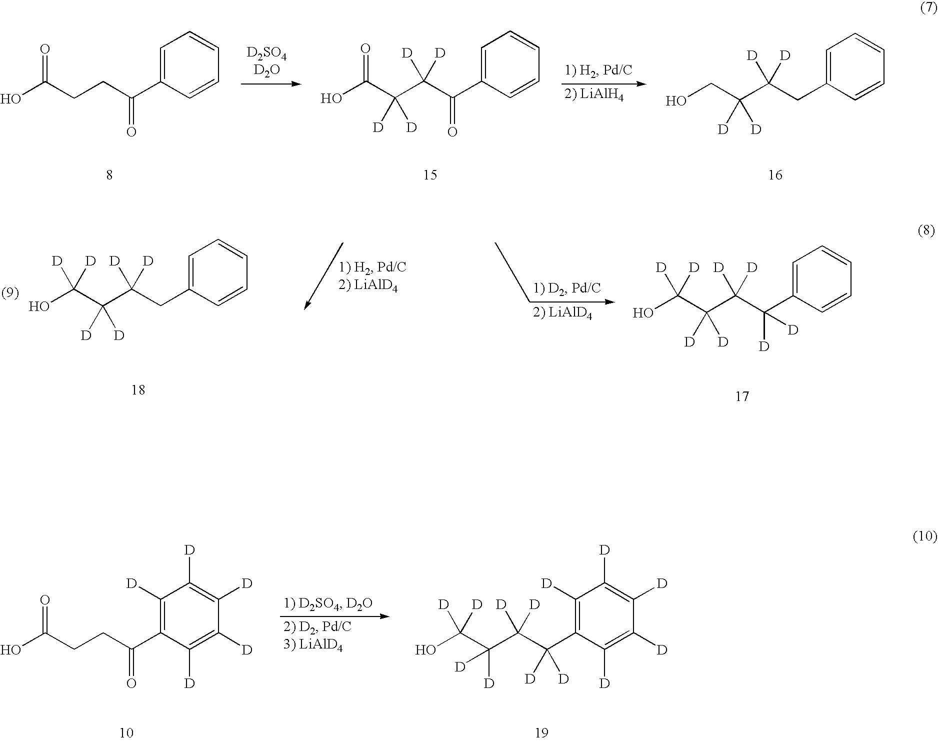

Scheme 3 shows additional deuterated starting materials and intermediates for the synthesis of deuterated forms of salmeterol. Various deuterated forms of the dibromide 2 from Scheme 1 are known and are shown in Scheme 3, i.e., commercially available 20 and the known compounds 21-24. If 20 is used in place of 2 in the chemistry of Scheme 1, salmeterol with R₁₃-R₂₄=D results. If 21 is used in place of 2 in the chemistry of Scheme 1, salmeterol with R₁₃-R₁₆ and R₂₁-R₂₄=D results. If 22 is used in place of 2 in the chemistry of Scheme 1, salmeterol with R₁₇-R₂₀=D results. If 23 is used in place of 2 in the chemistry of Scheme 1, salmeterol with R₁₃-R₁₄ and R₂₃-R₂₄=D results. If 24 is used in place of 2 in the chemistry of Scheme 1, salmeterol with R₁₅-R₁₆ and R₂₁-R₂₂=D results. Proton-deuterium exchange of 7 affords 25 as shown in equation (1) of Scheme 3. If 25 is used in place of 7 in the chemistry of Scheme 1, salmeterol with R₁₁-R₁₂=D results. Compound 5 can be made from compound 26 as shown in equation (2) of Scheme 3. Various deuterated forms of 26 are known, i.e., 27-31. If 27 is used in the chemistry of equation (2) of Scheme 3 and the resultant compound is used in place of 5 in the chemistry of Scheme 1, salmeterol with R₇-R₉=D results. If 28 is used in the chemistry of equation (2) of Scheme 3 and the resultant compound is used in place of 5 in the chemistry of Scheme 1, salmeterol with R₈-R₉=D results. If 29 is used in the chemistry of equation (2) of Scheme 3 and the resultant compound is used in place of 5 in the chemistry of Scheme 1, salmeterol with R₇=D results. If 30 is used in the chemistry of equation (2) of Scheme 3 and the resultant compound is used in place of 5 in the chemistry of Scheme 1, salmeterol with R₇ and R₉=D results. If 31 is used in the chemistry of equation (2) of Scheme 3 and the resultant compound is used in place of 5 in the chemistry of Scheme 1, salmeterol with R₈=D results. Reduction of 7 to trideuterated salmeterol 32 (R₅-R₆ and R₁₀=D) as shown in equation (3) is known (Molinski, et al., J. Label. Cpd. Radiopharm. 2002, 45, 755-762). This reduction sequence may be employed in combination with the above deuterated compounds to access even more salmeterol analogs. Again, a person skilled in the art of organic synthesis will recognize that the compounds and routes given above can be used in many combinations that are not shown to access deuterated salmeterols that are not shown. The chemistry shown is meant to be exemplary, not comprehensive.

### EXAMPLES

Table 1 provides compounds that are representative examples of the present invention. When one of R₁-R₃₇ is present, it is selected from H or D.

| | |
|---|---|
| 1 | 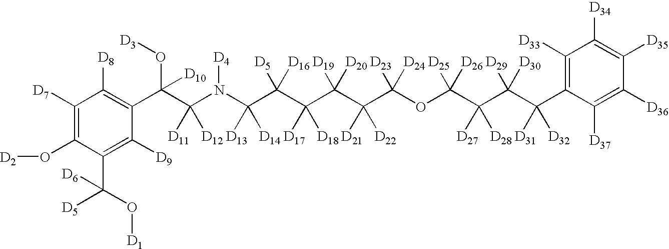 |
| 2 | 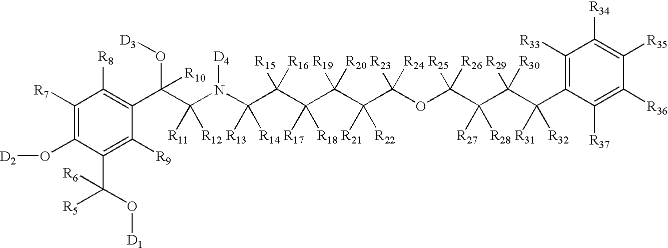 |
| 3 | 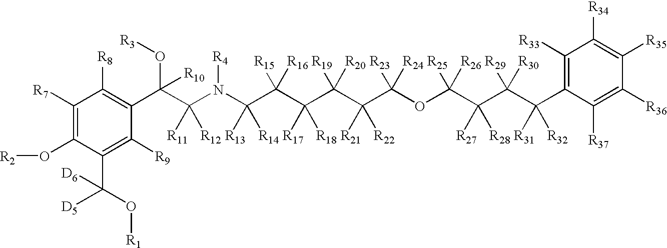 |
| 4 | 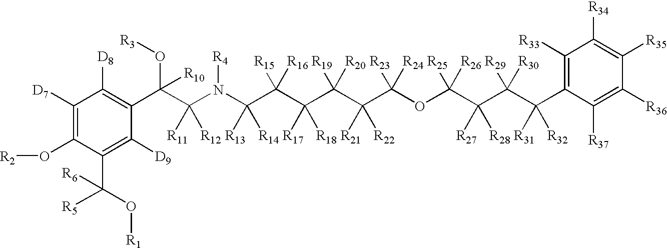 |
| 5 | 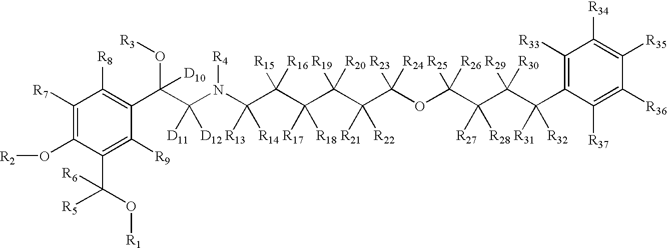 |
| 6 | 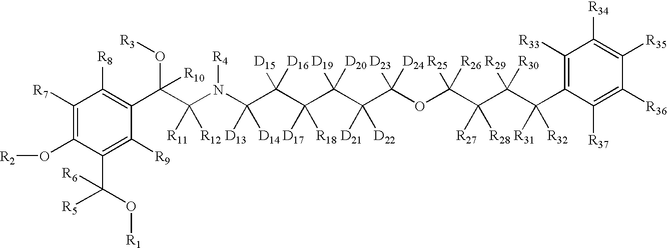 |
| 7 | 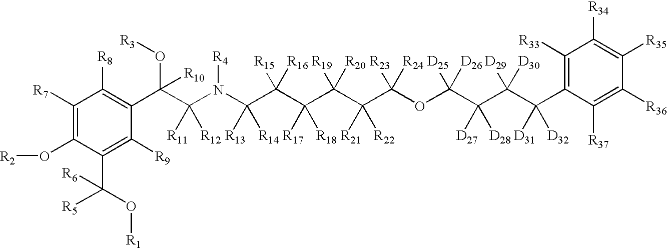 |
| 8 | 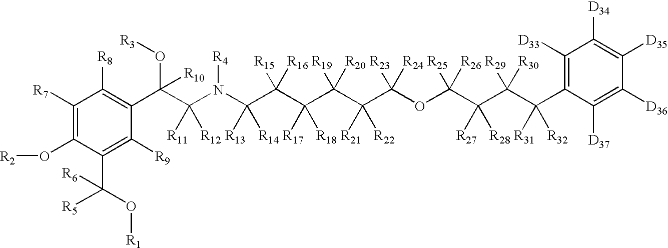 |

Table 2 provides compounds that are representative examples of the present invention. Where H is shown, it represents naturally abundant hydrogen.

| | |
|---|---|
| 9 | 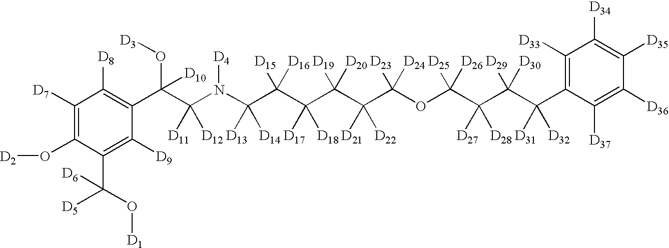 |
| 10 | 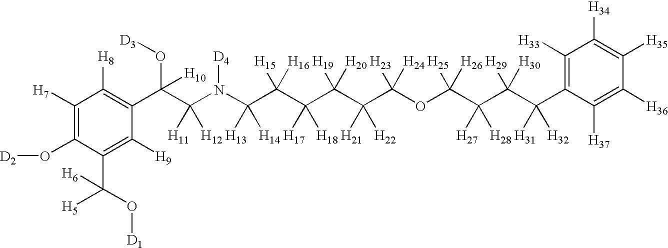 |
| 11 | 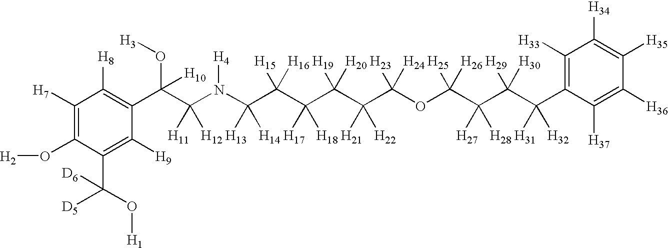 |
| 12 | 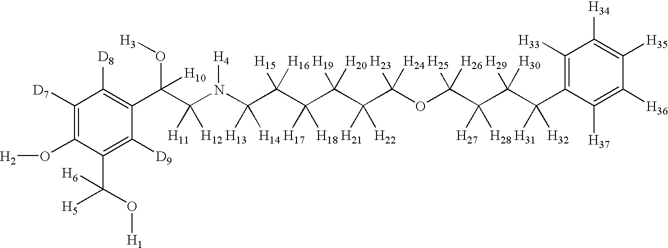 |
| 13 | 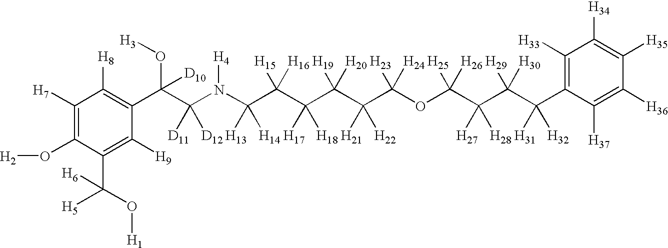 |
| 14 | 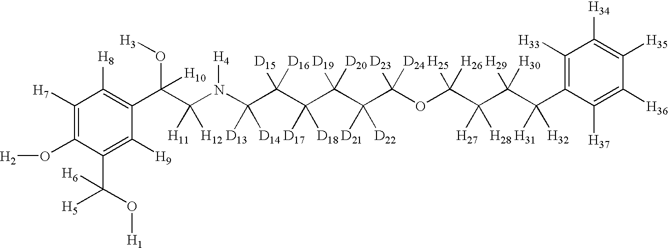 |
| 15 | 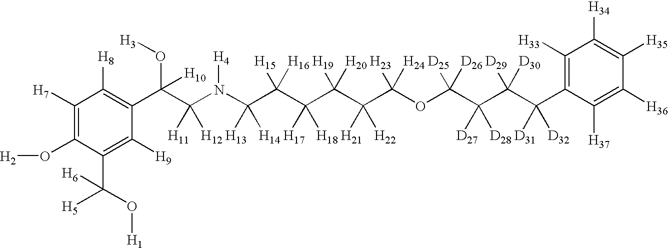 |
| 16 | 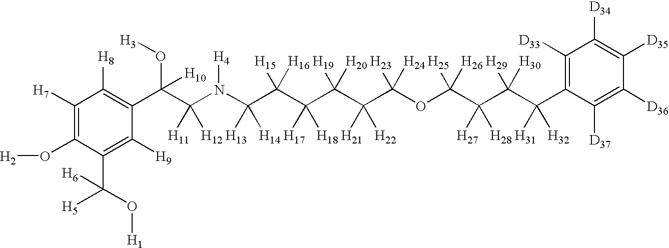 |

Numerous modifications and variations of the present invention are possible in light of the above teachings. It is therefore to be understood that within the scope of the appended claims, the invention may be practiced otherwise that as specifically described herein.

What is claimed is:

1. A deuterium-enriched compound of formula I or a pharmaceutically acceptable salt thereof: 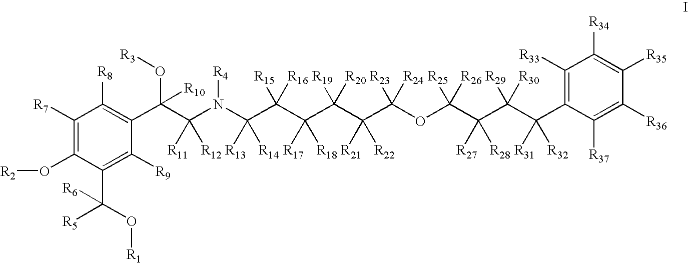
wherein R₁-R₃₇ are independently selected from H and D; and the abundance of deuterium in R₁-R₃₇ is at least 3%, provided that if R₅-R₆ and Rio are D, then at least one other R is a D.

2. A deuterium-enriched compound of claim 1, wherein the abundance of deuterium in R₁-R₃₇ is selected from at least 3%, at least 5%, at least 11%, at least 16%, at least 22%, at least 27%, at least 32%, at least 38%, at least 43%, at least 49%, at least 54%, at least 59%, at least 65%, at least 70%, at least 76%, at least 81%, at least 86%, at least 92%, at least 97%, and 100%.

3. A deuterium-enriched compound of claim 1, wherein the abundance of deuterium in R₁-R₄ is selected from at least 25%, at least 50%, at least 75%, and 100%.

4. A deuterium-enriched compound of claim 1, wherein the abundance of deuterium in R₅-R₆ is selected from at least 50% and 100%.

5. A deuterium-enriched compound of claim 1, wherein the abundance of deuterium in R₇-R₉ is selected from at least 33%, at least 67%, and 100%.

6. A deuterium-enriched compound of claim 1, wherein the abundance of deuterium in R₁₀-R₁₂ is selected from at least 33%, at least 67%, and 100%.

7. A deuterium-enriched compound of claim 1, wherein the abundance of deuterium in R₁₃-R₂₄ is selected from at least 8%, at least 17%, at least 25%, at least 33%, at least 42%, at least 50%, at least 58%, at least 67%, at least 75%, at least 83%, at least 92%, and 100%.

8. A deuterium-enriched compound of claim 1, wherein the abundance of deuterium in R₂₅-R₃₂ is selected from at least 13%, at least 25%, at least 38%, at least 50%, at least 63%, at least 75%, at least 88%, and 100%.

9. A deuterium-enriched compound of claim 1, wherein the abundance of deuterium in R₃₃-R₃₇ is selected from at least 20%, at least 40%, at least 60%, at least 80%, and 100%.

10. A deuterium-enriched compound of claim 1, wherein the compound is selected from compounds 1-8 of Table 1:

11. A deuterium-enriched compound of claim 1, wherein the compound is selected from compounds 9-16 of Table 2:

12. An isolated deuterium-enriched compound of formula I or a pharmaceutically acceptable salt thereof: 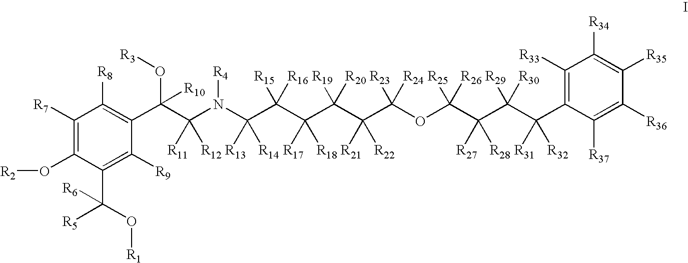
wherein R₁-R₃₇ are independently selected from H and D; and the abundance of deuterium in R₁-R₃₇ is at least 3%, provided that if R₅-R₆ and R₁₀ are D, then at least one other R is a D.

13. An isolated deuterium-enriched compound of claim 12, wherein the abundance of deuterium in R₁-R₃₇ is selected from at least 3%, at least 5%, at least 11%, at least 16%, at least 22%, at least 27%, at least 32%, at least 38%, at least 43%, at least 49%, at least 54%, at least 59%, at least 65%, at least 70%, at least 76%, at least 81%, at least 86%, at least 92%, at least 97%, and 100%.

14. An isolated deuterium-enriched compound of claim 12, wherein the abundance of deuterium in R₁-R₄ is selected from at least 25%, at least 50%, at least 75%, and 100%.

15. An isolated deuterium-enriched compound of claim 12, wherein the abundance of deuterium in R₅-R₆ is selected from at least 50% and 100%.

16. An isolated deuterium-enriched compound of claim 12, wherein the abundance of deuterium in R₇-R₉ is selected from at least 33%, at least 67%, and 100%.

17. An isolated deuterium-enriched compound of claim 12, wherein the abundance of deuterium in R₁₀-R₁₂ is selected from at least 33%, at least 67%, and 100%.

18. An isolated deuterium-enriched compound of claim 12, wherein the abundance of deuterium in R₁₃-R₂₄ is selected from at least 8%, at least 17%, at least 25%, at least 33%, at least 42%, at least 50%, at least 58%, at least 67%, at least 75%, at least 83%, at least 92%, and 100%.

19. An isolated deuterium-enriched compound of claim 12, wherein the abundance of deuterium in R₂₅-R₃₂ is selected from at least 13%, at least 25%, at least 38%, at least 50%, at least 63%, at least 75%, at least 88%, and 100%.

20. An isolated deuterium-enriched compound of claim 12, wherein the abundance of deuterium in R₃₃-R₃₇ is selected from at least 20%, at least 40%, at least 60%, at least 80%, and 100%.

21. An isolated deuterium-enriched compound of claim 12, wherein the compound is selected from compounds 1-8 of Table 1:

22. An isolated deuterium-enriched compound of claim 12, wherein the compound is selected from compounds 9-16 of Table 2:

23. A mixture of deuterium-enriched compounds of formula I or a pharmaceutically acceptable salt thereof: 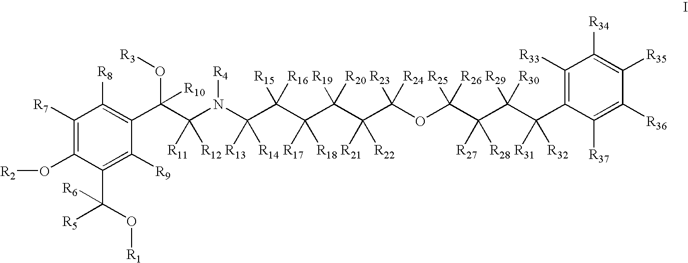
wherein R₁-R₃₇ are independently selected from H and D; and the abundance of deuterium in R₁-R₃₇ is at least 3%, provided that if R₅-R₆ and R₁₀ are D, then at least one other R is a D.

24. A mixture of deuterium-enriched compounds of claim 23, wherein the abundance of deuterium in R₁-R₃₇ is selected from at least 3%, at least 5%, at least 11%, at least 16%, at least 22%, at least 27%, at least 32%, at least 38%, at least 43%, at least 49%, at least 54%, at least 59%, at least 65%, at least 70%, at least 76%, at least 81%, at least 86%, at least 92%, at least 97%, and 100%.

25. A mixture of deuterium-enriched compounds of claim 23, wherein the abundance of deuterium in R₁-R₄ is selected from at least 25%, at least 50%, at least 75%, and 100%.

26. A mixture of deuterium-enriched compounds of claim 23, wherein the abundance of deuterium in R₅-R₆ is selected from at least 50% and 100%.

27. A mixture of deuterium-enriched compounds of claim 23, wherein the abundance of deuterium in R₇-R₉ is selected from at least 33%, at least 67%, and 100%.

28. A mixture of deuterium-enriched compounds of claim 23, wherein the abundance of deuterium in R₁₀-R₁₂ is selected from at least 33%, at least 67%, and 100%.

29. A mixture of deuterium-enriched compounds of claim 23, wherein the abundance of deuterium in R₁₃-R₂₄ is selected from at least 8%, at least 17%, at least 25%, at least 33%, at least 42%, at least 50%, at least 58%, at least 67%, at least 75%, at least 83%, at least 92%, and 100%.

30. A mixture of deuterium-enriched compounds of claim 23, wherein the abundance of deuterium in R₂₅-R₃₂ is selected from at least 13%, at least 25%, at least 38%, at least 50%, at least 63%, at least 75%, at least 88%, and 100%.

31. A mixture of deuterium-enriched compounds of claim 23, wherein the abundance of deuterium in R₃₃-R₃₇ is selected from at least 20%, at least 40%, at least 60%, at least 80%, and 100%.

32. A mixture of deuterium-enriched compounds of claim 23, wherein the compound is selected from compounds 1-8 of Table 1:

33. A mixture of deuterium-enriched compounds of claim 23, wherein the compound is selected from compounds 9-16 of Table 2:

34. A pharmaceutical composition, comprising: a pharmaceutically acceptable carrier and a therapeutically effective amount of a compound of claim 1 or a pharmaceutically acceptable salt form thereof.

35. A method for treating asthma comprising: administering, to a patient in need thereof, a therapeutically effective amount of a compound of claim 1 or a pharmaceutically acceptable salt form thereof.
